(19) United States
(12) Patent Application Publication
(10) Pub. No.: US 2024/0081936 A1
(43) Pub. Date: Mar. 14, 2024

(54) MEDICAL PROCEDURE KIT AND ASSOCIATED METHODS

(71) Applicant: Medline Industries, LP, Northfield, IL (US)

(72) Inventors: Kelly FitzPatrick Nace, Slingerlands, NY (US); Bernadette Cremin, Fairfield, CT (US); Bethanie Szydlowski, Columbia, TN (US); Brianna Sarley, Chicago, IL (US); Amy Olson, Indianapolis, IN (US)

(21) Appl. No.: 17/944,733

(22) Filed: Sep. 14, 2022

(51) Int. Cl. A61B 50/33

(52) U.S. Cl. CPC A61B 50/33; A61B 2050/3008

(57) ABSTRACT

A medical procedure kit and related methods are provided. The medical procedure kit includes a tray defining a plurality of compartments, a medical waste basin removably supported within a first compartment of the plurality of compartments, and a plurality of medical procedure tools located within other compartments of the plurality of compartments. Additionally, the first compartment defines an access opening to manually engage the medical waste basin.

### TECHNICAL FIELD

The present disclosure relates to a medical procedure kit and associated methods. In particular, the present disclosure relates to a medical procedure kit that includes a removable medical waste basin.

### BACKGROUND

Use of procedure specific medical kits containing all the various medical tools needed for the procedure are known and are specifically useful in increasing procedure efficiency and reducing medical errors and other complications such as patient infections. Some known kits can include a tray that contains and lays out the tools needed for the procedure. Additionally, some of these known tray-based kits include an additional compartment in the tray into which biological material from the procedure can be deposited. In certain instances, the biological material is biohazard material that requires special and expensive disposal procedures of the entire tray.

### BRIEF DESCRIPTION OF THE DRAWINGS

FIG. 1 is a top view of a tray according to disclosed embodiments;

FIG. 2 is a bottom view of a tray according to disclosed embodiments;

FIG. 3 is front side view of a tray according to disclosed embodiments;

FIG. 4 is a left side view of a tray according to disclosed embodiments;

FIG. 5 is a back side view of a tray according to disclosed embodiments;

FIG. 6 is a right side view of a tray according to disclosed embodiments;

FIG. 7 is a perspective view of a medical waste basin according to disclosed embodiments;

FIG. 8A is a perspective view of a side of a medical waste basin according to disclosed embodiments;

FIG. 8B is a perspective view of a side of a medical waste basin according to disclosed embodiments;

FIG. 9 is a perspective view of a medical waste basin in a tray according to disclosed embodiments;

FIG. 10 is a perspective view of a medical waste basin in a tray according to disclosed embodiments;

FIG. 11A is a perspective view of a medical waste basin in a tray according to disclosed embodiments;

FIG. 11B is a perspective view of a medical waste basin in a tray according to disclosed embodiments;

FIG. 12A is a cross-sectional view along line A-A in FIG. 11B of a medical waste basin in a tray according to disclosed embodiments;

FIG. 12B is a cross-sectional view along line B-B in FIG. 11B of a medical waste basin in a tray according to disclosed embodiments;

FIG. 12C is a cross-sectional view along line C-C in FIG. 11B of a medical waste basin in a tray according to disclosed embodiments;

FIG. 13 is a top view of a compartment for a medical waste basin in a tray according to disclosed embodiments;

FIG. 14 is a perspective view of a compartment for a medical waste basin in a tray according to disclosed embodiments;

FIG. 15 is a top view of a compartment for a medical waste basin in a tray according to disclosed embodiments;

FIG. 16 is a perspective view of a medical procedure kit according to disclosed embodiments;

FIG. 17 is a perspective view of an opened medical procedure kit according to disclosed embodiments;

FIG. 18 is a top view of medical procedure tools included in a medical procedure kit according to disclosed embodiments;

FIG. 19 is a top view of sterile wrapped parts included in a medical procedure kit according to disclosed embodiments;

FIG. 20 is a perspective view of medical procedure tools included in sterile wrapped parts of a medical procedure kit according to disclosed embodiments;

FIG. 21 is a front view of a gown included in a medical procedure kit according to disclosed embodiments;

FIG. 22 is a close-up view of a gown included in a medical procedure kit according to disclosed embodiments;

FIG. 23 is a close-up view of a gown included in a medical procedure kit according to disclosed embodiments;

FIG. 24 is a perspective view of patient preparation tools in a medical procedure kit according to disclosed embodiments;

FIG. 25 is a close-up view of a cleansing swab included in a medical procedure kit according to disclosed embodiments;

FIG. 26 is a front view of a drape included in a medical procedure kit according to disclosed embodiments;

FIG. 27 is a close-up view of a drape included in a medical procedure kit according to disclosed embodiments;

FIG. 28 is a closeup view of a drape included in a medical procedure kit according to disclosed embodiments;

FIG. 29 is a perspective view of a tray with a plurality of medical procedure tools included in a medical procedure kit according to disclosed embodiments;

FIG. 30 is a perspective view of ultrasound tools included in a medical procedure kit according to disclosed embodiments;

FIG. 31 is a close-up view of ultrasound tools included in a medical procedure kit according to disclosed embodiments;

FIG. 32 is a perspective view of a bottom of an ultrasound sleeve included in a medical procedure kit according to disclosed embodiments;

FIG. 33 is a perspective view of a top of an ultrasound sleeve included in a medical procedure kit according to disclosed embodiments;

FIG. 34 is a perspective view of an ultrasound sleeve included in a medical procedure kit according to disclosed embodiments;

FIG. 35 is a top view of a tray with a plurality of medical procedure tools included in a medical procedure kit according to disclosed embodiments;

FIG. 36 is a close-up top view of a portion of a tray included in a medical procedure kit according to disclosed embodiments;

FIG. 37 is a top view of a vial content indicator included in a medical procedure kit according to disclosed embodiments;

FIG. 38 is a perspective view of syringes included in a medical procedure kit according to disclosed embodiments;

FIG. 39 is a perspective view of a vessel dilator included in a medical procedure kit according to disclosed embodiments;

FIG. 40 is a top view of a catheter guide wire included in a medical procedure kit according to disclosed embodiments;

FIG. 41 is a perspective view of a hook of a catheter guide wire included in a medical procedure kit according to disclosed embodiments;

FIG. 42 is a close-up view of a catheter guide wire included in a medical procedure kit according to disclosed embodiments;

FIG. 43 is a perspective view of a catheter included in a medical procedure kit according to disclosed embodiments;

FIG. 44 is a close-up view of a needle-less connector included in a medical procedure kit according to disclosed embodiments;

FIG. 45 is a close-up view of a needle-less connector included in a medical procedure kit according to disclosed embodiments;

FIG. 46 is a close up view of a needle-less connector included in a medical procedure kit according to disclosed embodiments;

FIG. 47 is a perspective view of a suture loop included in a medical procedure kit according to disclosed embodiments;

FIG. 48 is a perspective view of a box clamp included in a medical procedure kit according to disclosed embodiments;

FIG. 49 is top view of a needle vise and a needle receptacle included in a medical procedure kit according to disclosed embodiments;

FIG. 50 is a front perspective view of a needle vise included in a medical procedure kit according to disclosed embodiments; and

FIG. 51 is a top view of suture tools included in a medical procedure kit according to disclosed embodiments.

### DETAILED DESCRIPTION

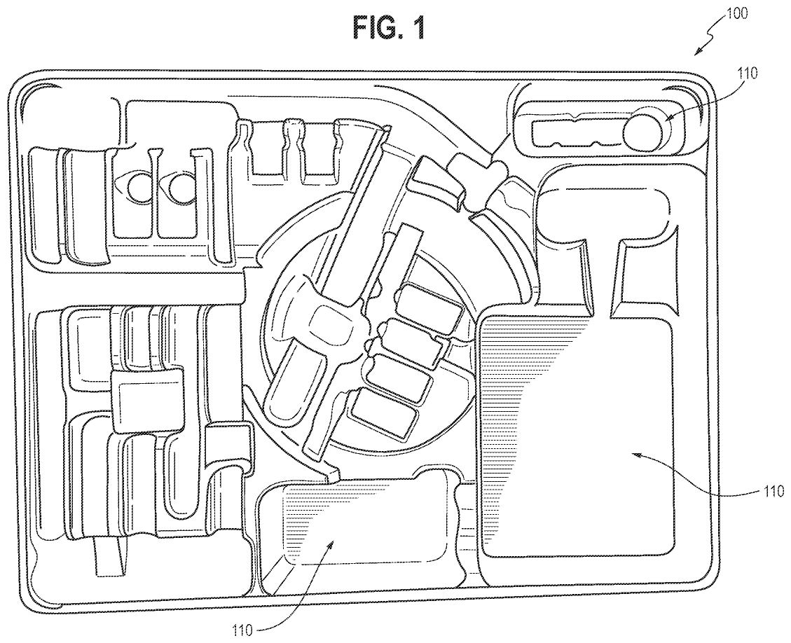

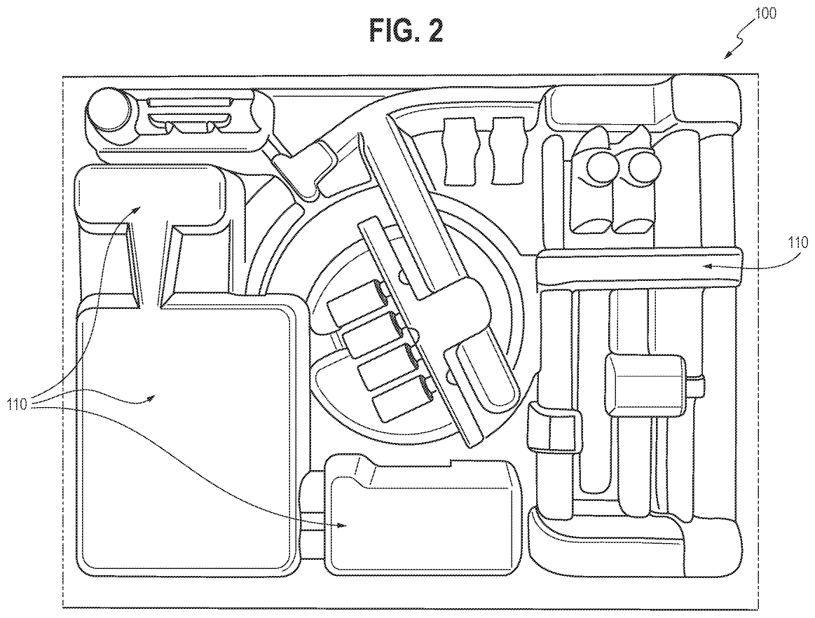

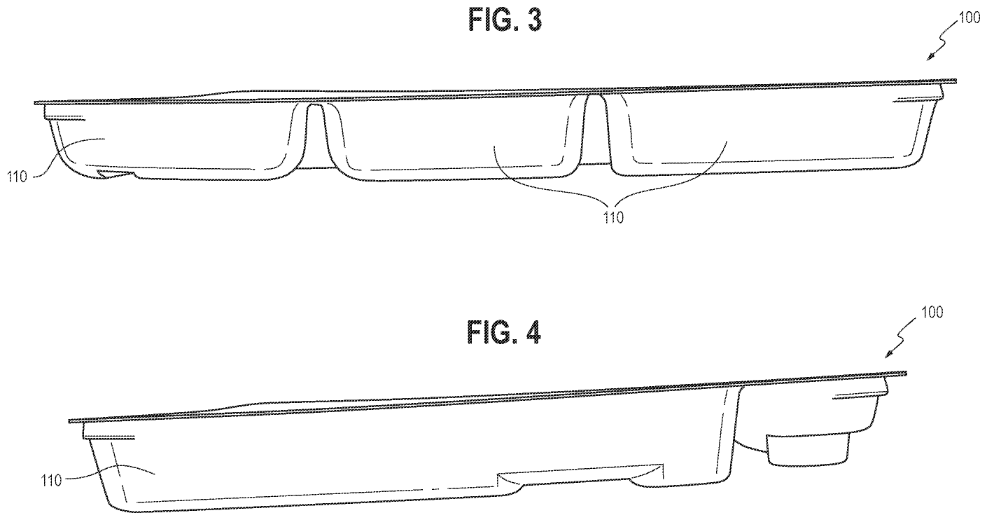

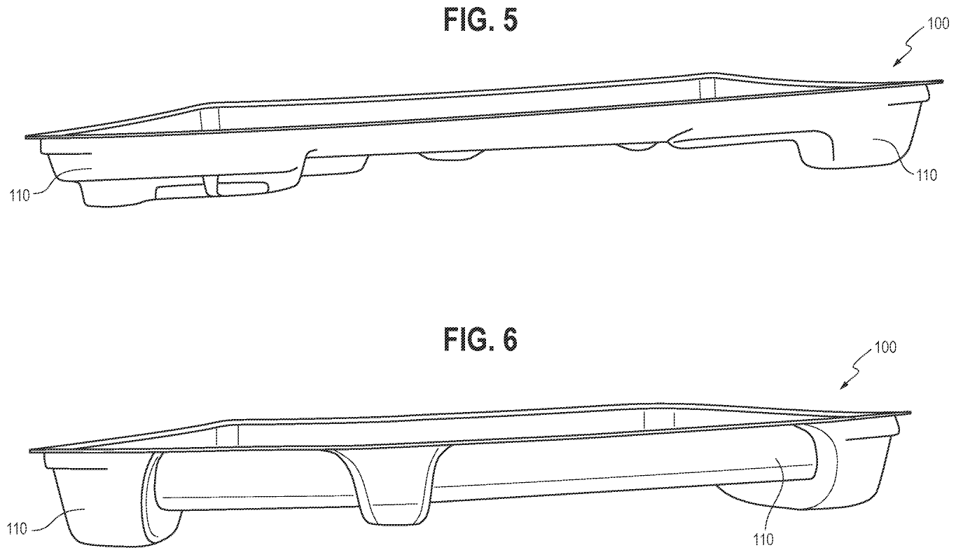

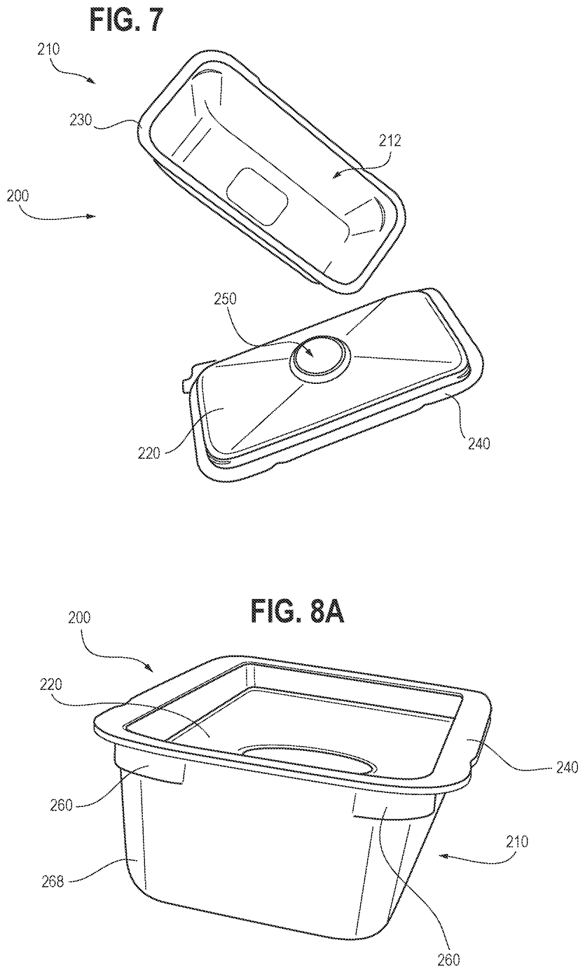

Embodiments described herein are directed to a medical procedure kit that includes a tray 100 (See FIGS. 1-6) and a medical waste basin 200 (see FIGS. 7-12). A top view, a bottom view, front side view, a left side view, a back side view, and a right side view of the tray 100 are shown in FIGS. 1, 2, 3, 4, and 5 respectively. As seen in FIGS. 1-5, the tray 100 can define a plurality of compartments 110. As discussed in more detail below, the plurality of compartments 110 can have various sizes and shapes to accommodate the medical waste basin 200 (see FIGS. 7-12), as well as a plurality of medical procedure tools. In some embodiments, the plurality of compartments 110 can be integrally formed as single piece construction to form the tray 100. For example, in some embodiments, the tray 100 and the plurality of compartments 110 can be made from a plastic material formed via a single manufacturing process, such as a vacuform process, an injection molding process, a quick setting mold process, and/or other known processes.

Referring now to FIG. 7, a perspective view of the medical waste basin 200 is shown. The medical waste basin 200 can include a body 210 that defines an interior region 212 for containing biological material separately from the tray 100. The medical waste basin 200 also can include a lid 220 with a first lip 240. The lid can define an aperture 250 sized to receive medical waste. For example, the size may be such that the aperture 250 receives at least a portion of at least one of the plurality of medical procedure tools from the tray 100. Further, the lid 220 can include a section that slopes toward the aperture 250 to assist in directing biological material to and through the aperture 250 into the interior region 212.

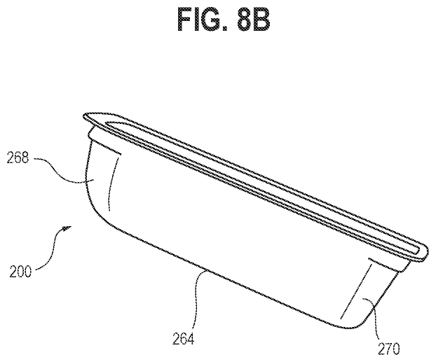

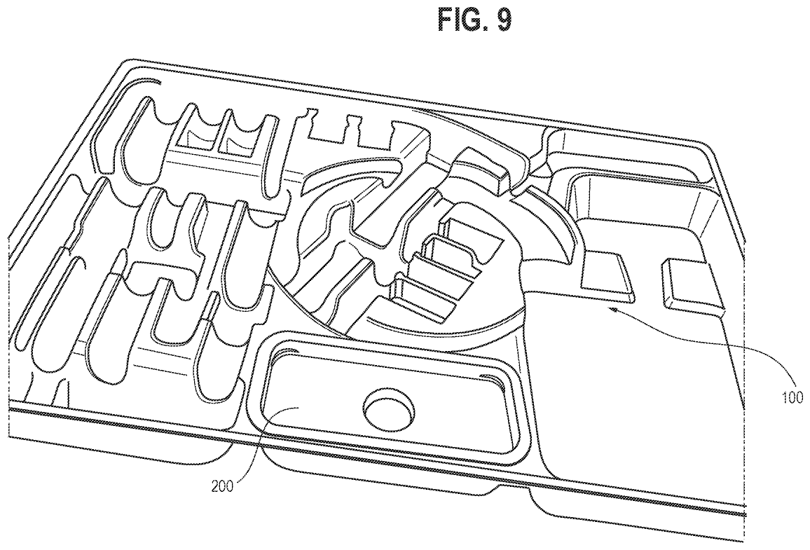

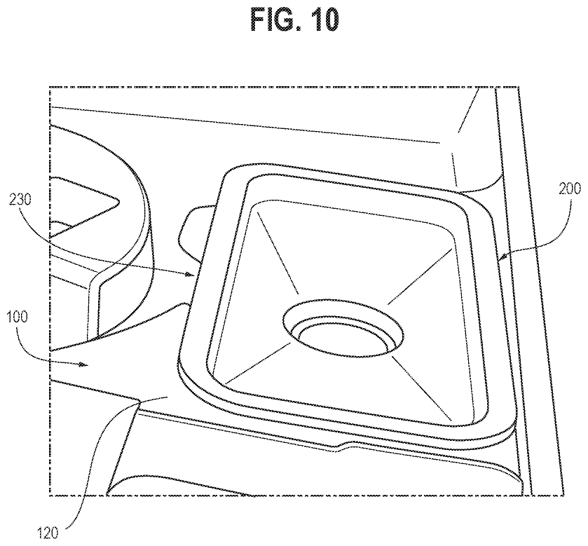

FIG. 8A is a perspective view of a short side 268 of the medical waste basin 200. As seen in FIG. 8A, the lid 220 can removably attach to the medical waste basin 200. In some embodiments, the first lip 240 can mate with a second lip 230 (see FIG. 7) to removably attach the lid 220 to the medical waste basin 200. Further, as seen in FIG. 8A, the medical waste basin 200 can include a plurality of protrusions 260 that engage with side walls 134 (See FIG. 13) of a first compartment 111 (see FIG. 13) of the plurality of compartments 110. The plurality of protrusions 260 can create a friction force against the side walls 134 to secure the medical waste basin 200 within the first compartment 111. In some embodiments, the protrusion 260 can form indentations in the side walls 134 of the first compartment 111. The plurality of protrusions 260 can be positioned under the second lip 230 and at corners of the medical waste basin 200.

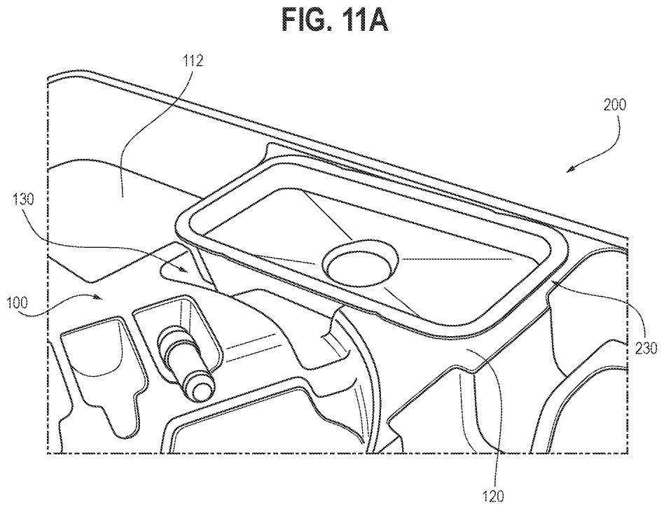

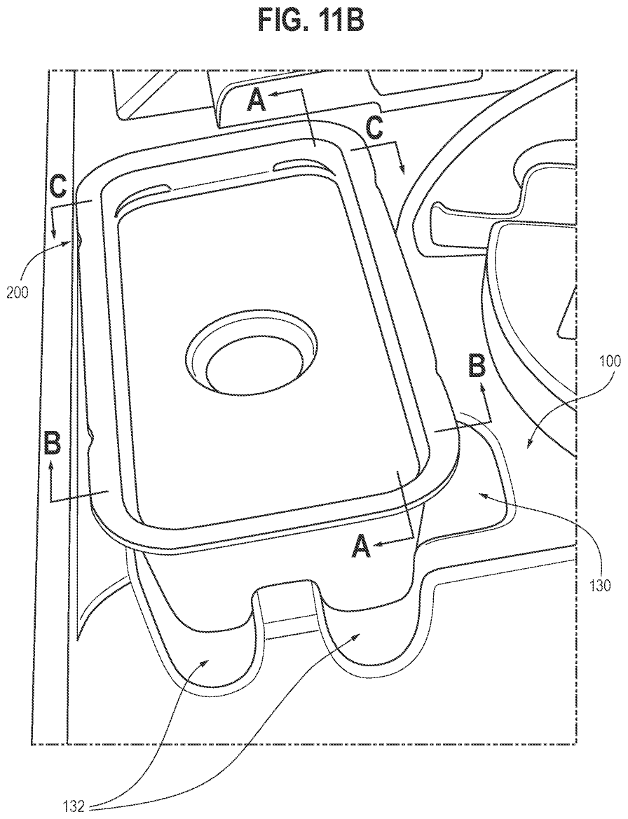

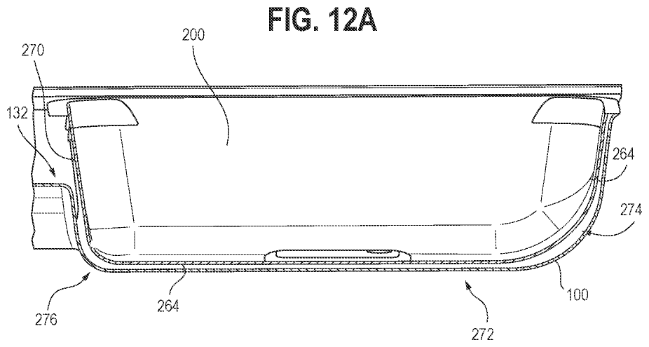

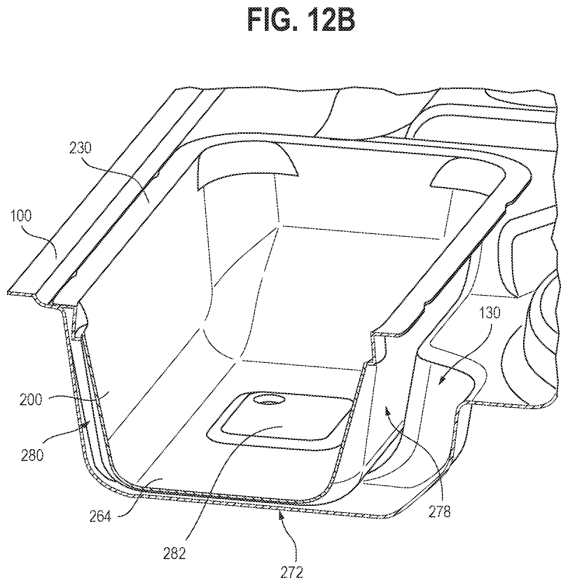

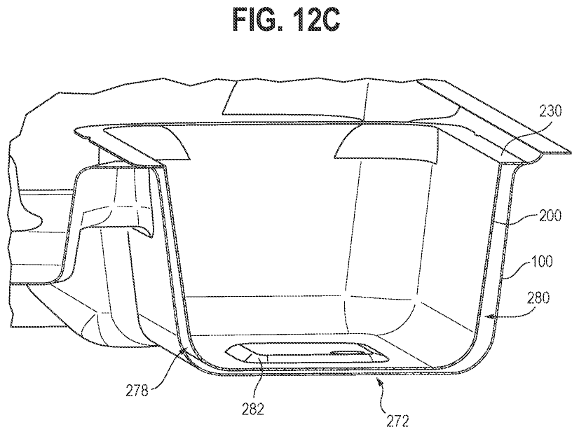

FIG. 8B is a perspective view of a long side of the medical waste basin 200. The medical waste basin 200 can include a bottom 264 and a plurality of side walls extending from the bottom 264. As noted above, the first side wall 268 of the plurality of sidewalls can be shorter than all other of the plurality of sidewalls, including a second side wall 270. Further still, in some embodiments, the first side wall 268 can have a larger radius of curvature when transitioning between the bottom 264 as compared to the transition between the other of the plurality of side walls and the bottom 264. Additionally, the second side wall 270 can be located adjacent an passages 132 (see FIGS. 12A-C) to the first compartment 111, such as from a neighboring compartment 112 (See FIG. 11A) of the tray 100 so as to increase the accessibility to the medical waste basin 200, such as for removal from the tray 100.

FIGS. 9 and 10 are perspective views of the medical waste basin 200 nested in the tray 100 according to disclosed embodiments. In some embodiments, the second lip 230 can rest on and be supported by webbing 120 of the tray between adjacent compartments 110. Like the tray 100, the medical waste basin 200 can be made from a plastic material formed via a vacuform process, an injection molding process, a quick setting mold process, and/or other similar process known in the art.

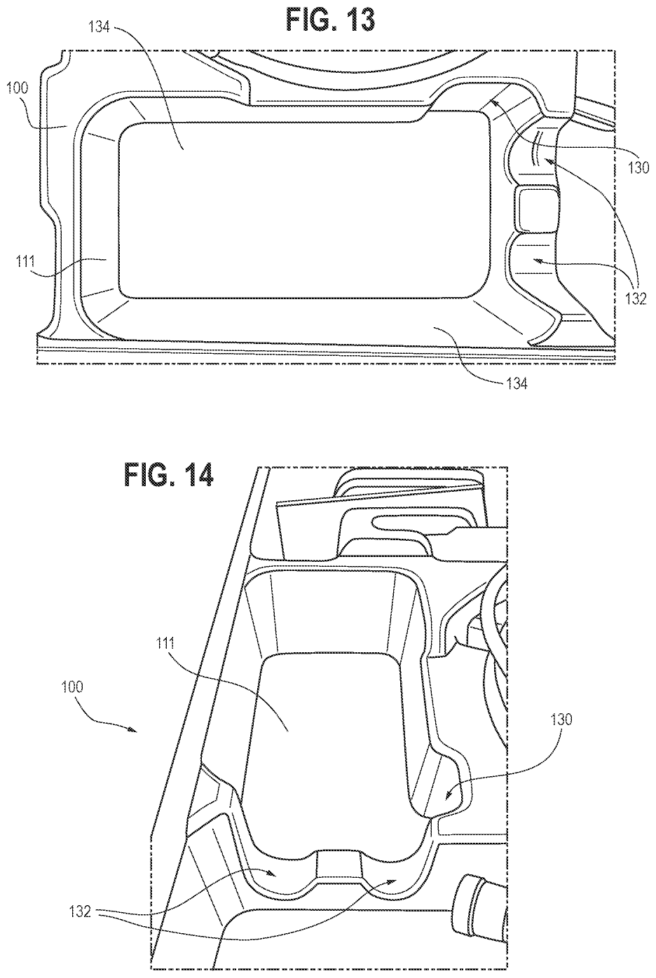

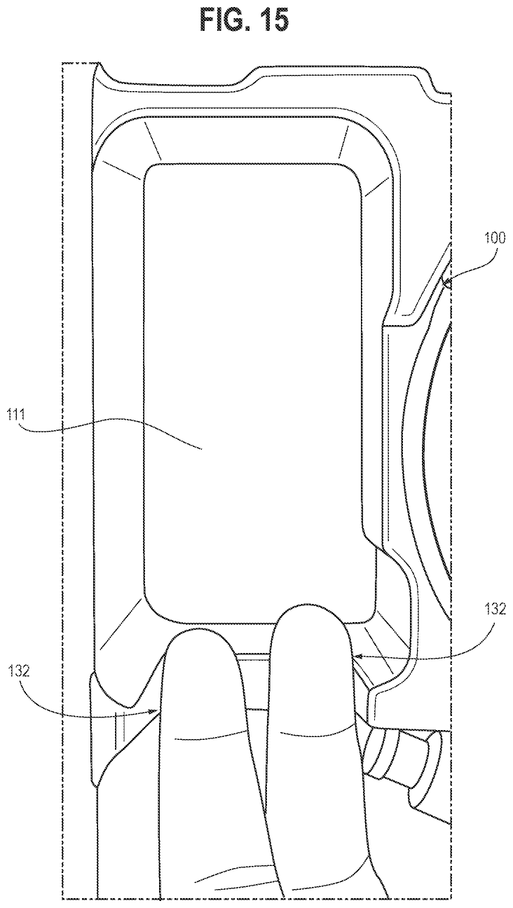

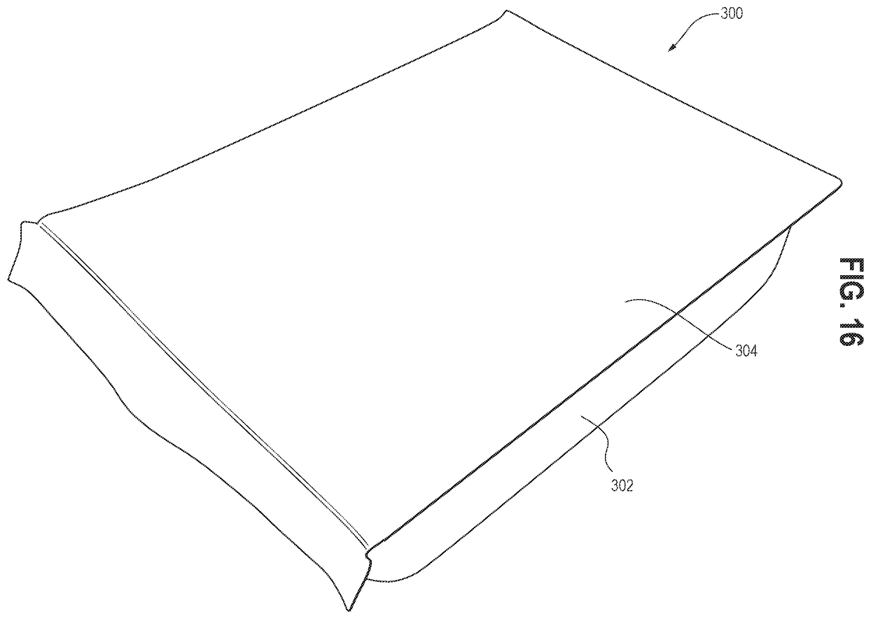

FIGS. 11A and 11B contain additional perspective views of the medical waste basin 200 nested in the tray 100 and FIGS. 12A-12C contain cross-sectional views of the medical waste basin 200 nested in the tray 100. FIGS. 13 and 14 contain a top and perspective views of the first compartment 111 for containing the medical waste basin 200 in the tray 100. As seen in FIGS. 11-14, the access opening 129 can be defined in the tray 100 to enable manual engagement with the medical waste basin 200. In some embodiments, the access opening 129 can include a passage 132 extending from the first compartment 111 (see FIGS. 13 and 14) to a neighboring compartment of the plurality of compartments 110. Further, the access opening 129 can include a lateral recess 130 that enables access to the medical waste basin 200 from above the tray 100. For example, the lateral recess 130 can enable access to the underside of the second lip 230 for easy removal of the medical waste basin 200 from the tray 100. The lateral recess 130 and passages 132 also can provide access to the lid 220 to enable its removal without taking the medical waste basin 130 out of the tray 100. As can be seen in FIG. 15, the passage 132 can be sized and shaped to enable a user's fingers to pass into the first compartment 111 and under the medical waste basin 200 so that the medical waste basin 200 can be removed from the tray 100 via the bottom 264 of the medical waste basin 200.

Turning again to FIG. 12A, when the medical waste basin 200 is nested within the tray 100, the bottom 264 can be spaced from a bottom of the tray 100 to form a first gap 272. Likewise, as discussed above, the first side wall 268 has a larger radius of curvature when transitioning between the bottom 264 as compared to the transition between the front side wall 270 and the bottom 264. Further, a second gap 274 is formed between the first side wall 268 and the tray 100, and a third gap 276 is formed between the front side wall 270 and the tray 100.

As seen in FIGS. 12B and 12C, a fourth gap 278 and a fifth gap 280 can also be formed between the side walls of the medical waste basin 200 and the tray 100. It will be appreciated that each of the gaps 272, 274, 276, and 280 can comprise a single unified space formed between the medical waste basin 200 and the tray 100. The gaps 272, 274, 276, and 280 also can have the same thickness, different thicknesses, or varying thicknesses. Alternatively, the outer profile of the medical waste basin 200 can, in whole or part, conform to the interior surface of the first compartment 111. Further, as seen in FIGS. 12A-12C, in some embodiments, the medical waste basin 200 can include a raised section 282 that protrudes up from the bottom 264.

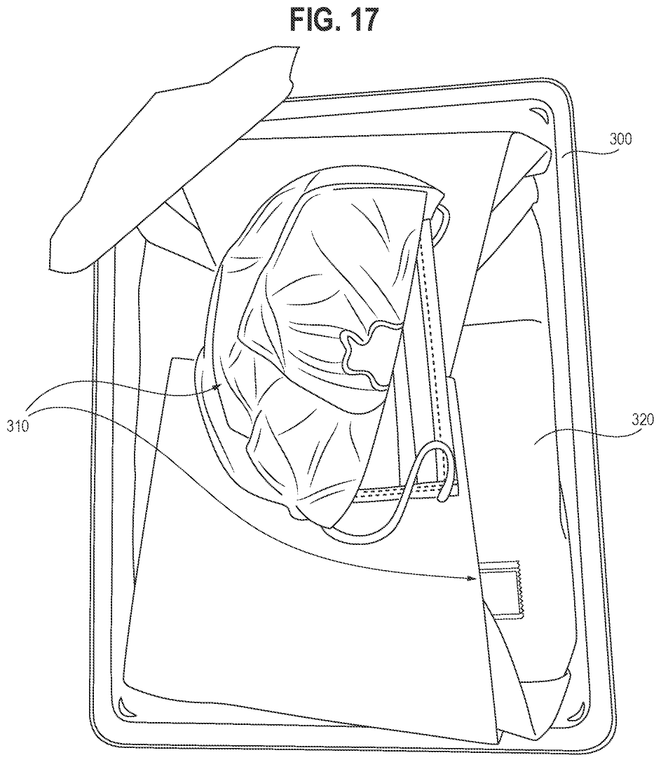

Turning now to FIG. 16, a perspective view of a medical procedure kit 300 that includes the tray 100 and the medical waste basin 200 is shown. The medical procedure kit 300 can include a container 302 and a sealed closure 304. As seen in FIG. 17, when the sealed closure 304 is removed, a portion of the medical procedure kit 300 can be seen. More specially, on can see first procedure tools 310 and a sterile wrap 320 that contains additional components of the medical procedure kit 300, such as the tray 100 and the medical waste basin 200.

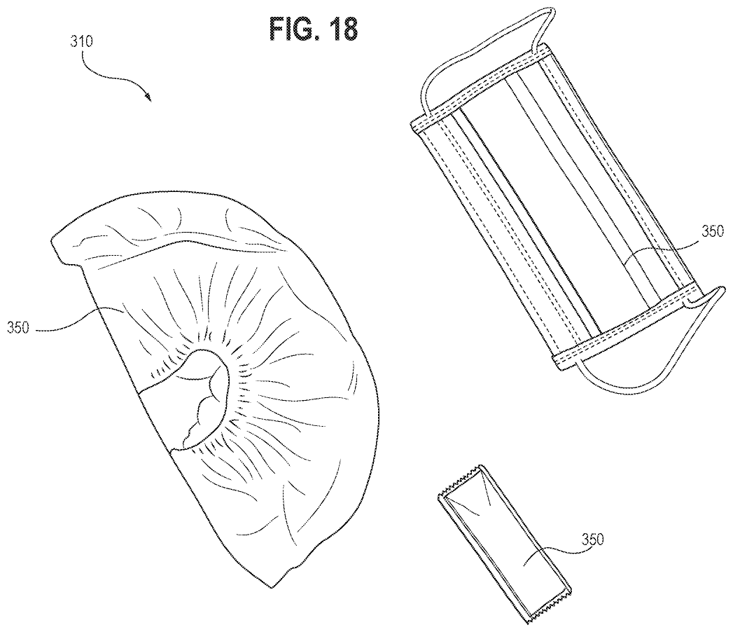

A top view of the first procedure tools 310 is shown in FIG. 18. The first procedure tools 310 can include a bouffant cap 330, a face mask 340 with a plastic guard, and a sanitizer packet 350.

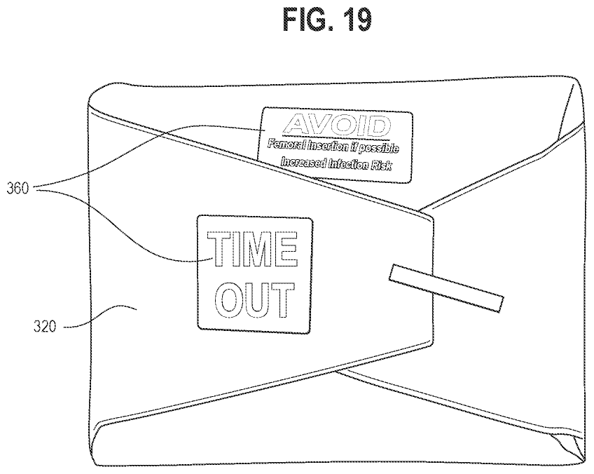

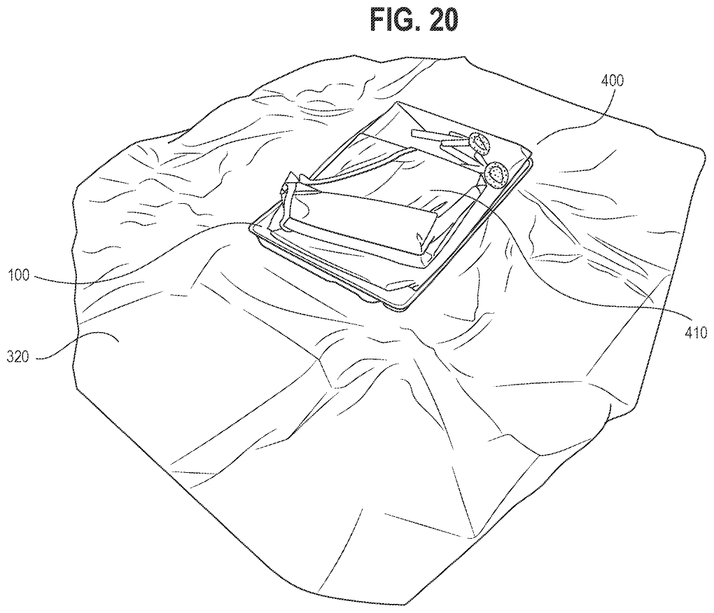

FIG. 19 is a top view of the sterile wrap 320 surrounding the additional components of the medical procedure kit 300 after being removed from the container 302. The sterile wrap 320 can include medical procedure guidance indicators 360 located on an exterior of the sterile wrap 320. For instance, the medical procedure guidance indicators 360 can caution a medical practitioner to proceed carefully so to perform the procedure in a focused and effective manner. Further, the medical procedure guidance indicators 360 can include specific guidance regarding the procedure, such as a reminder to avoid using femoral insertion of a catheter because of increased infection risk.

FIG. 20 is a perspective view of the medical procedure tools included within the sterile wrap 320. Those medical procedure tools can include the tray 100 and the plurality of tools contained therein, patient preparation tools 400, and a gown 410.

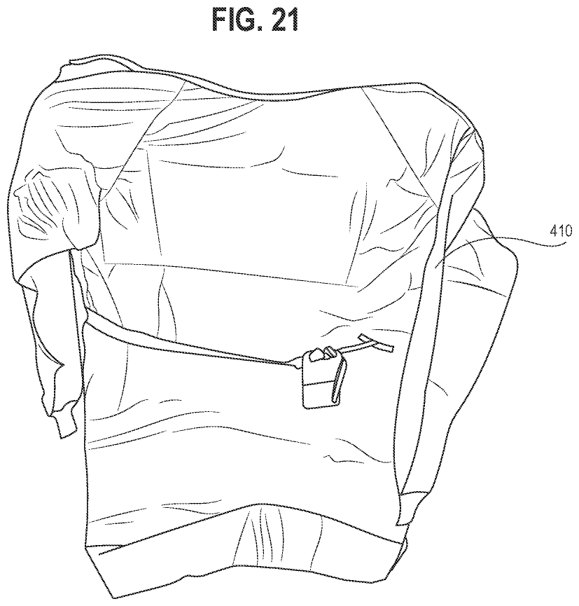

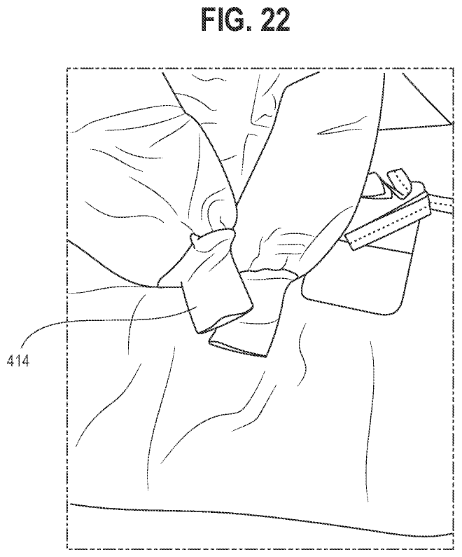

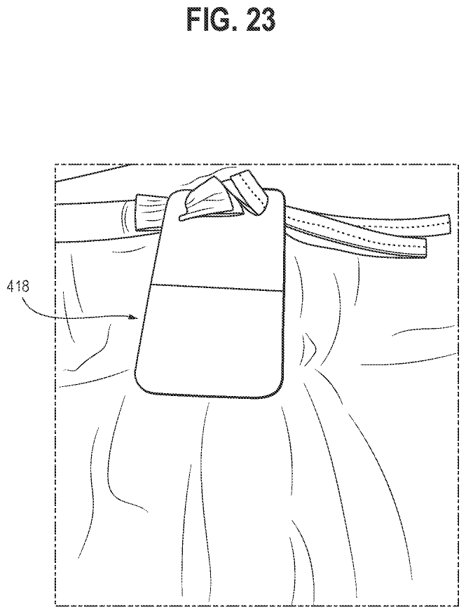

FIG. 21 is a front view of the gown 410, and FIGS. 22 and 23 are close-up views of the gown 410. The gown 410 can include cuffs 414 and a tie securing tab 418. When used as part of a procedure, an assistant can hold up the gown 410 for a practitioner to walk into the cuffs 414 via an open back of the gown 410. Then, the assistant can pull the tie securing tab 418 to release tie backs for the gown 410, which can then be tied around the practitioner to secure the gown 410.

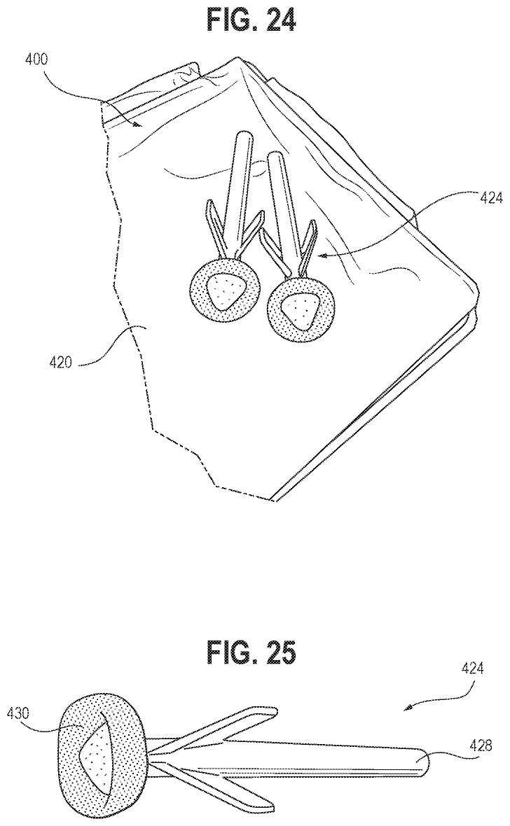

FIG. 24 is a perspective view of patient preparation tools 400. The patient preparation tools 400 can include a surgical drape 420 and medical cleansing swabs 424. A close-up view of one of the medical cleansing swabs 424 is shown in FIG. 25. The medical cleansing swabs 424 can include a body 428 that contains chlorhexidine gluconate (CHG). In some embodiments, a glass ampoule containing the CHG can be contained in the body 428. Further, the medical cleansing swabs 424 can include a foam tip 430 for applying the CHG to a patient. In some embodiments, the foam tip 430 can include an orange dye that helps the practitioner see that the CHG has been released and is wetting the foam tip 430.

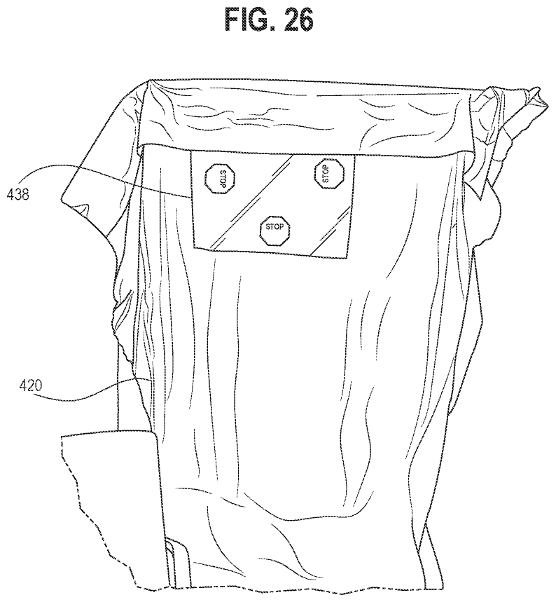

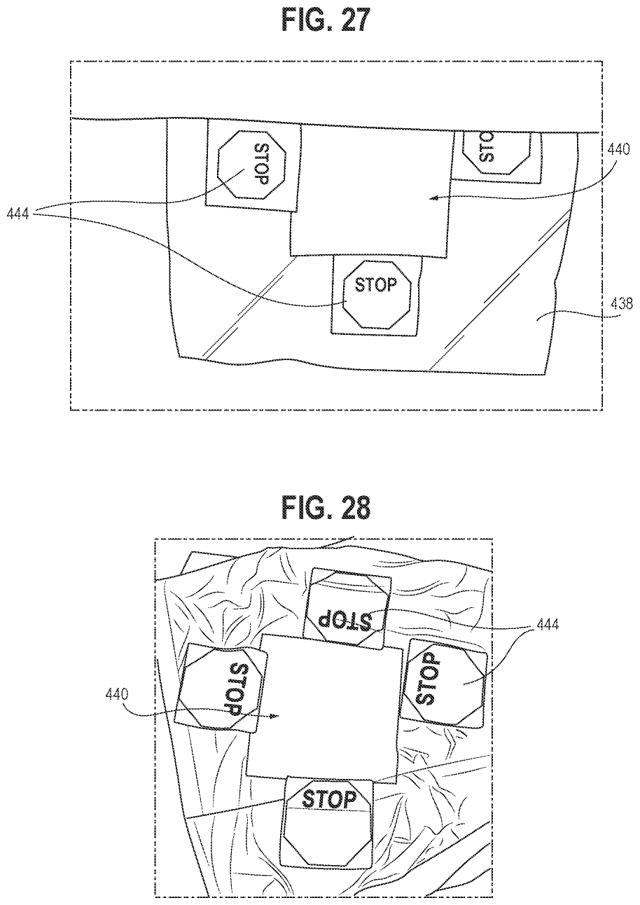

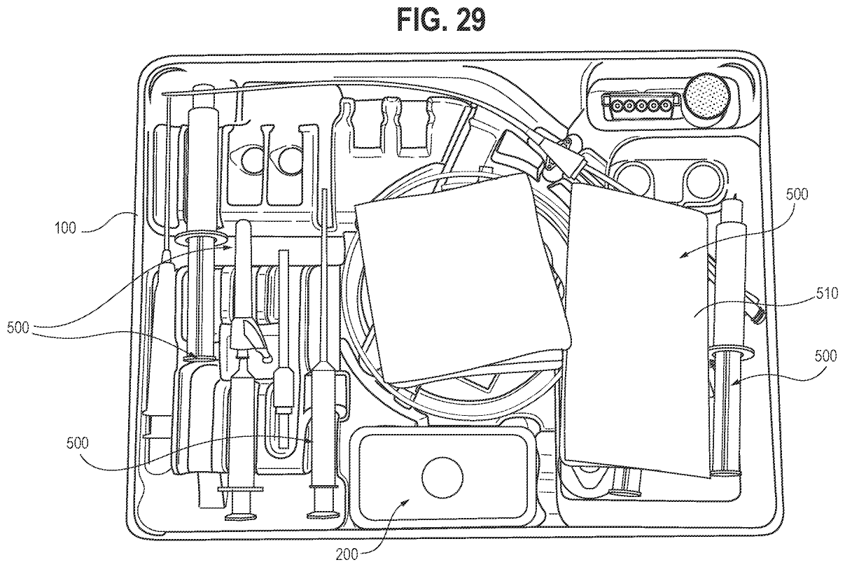

FIG. 26 is a front view of the surgical drape 420, and FIGS. 27 and 28 are close-up views of the surgical drape 420. As seen in FIG. 26, the surgical drape 420 can include a patient access area 438. As seen in FIGS. 27 and 28, the patient access area 438 can include a treatment area opening 440 surrounded by additional medical procedure guidance indicators 444. In some embodiments, the additional medical procedure guidance indicators 444 can remind the practitioner to remove a catheter insertion guide wire from a patient.

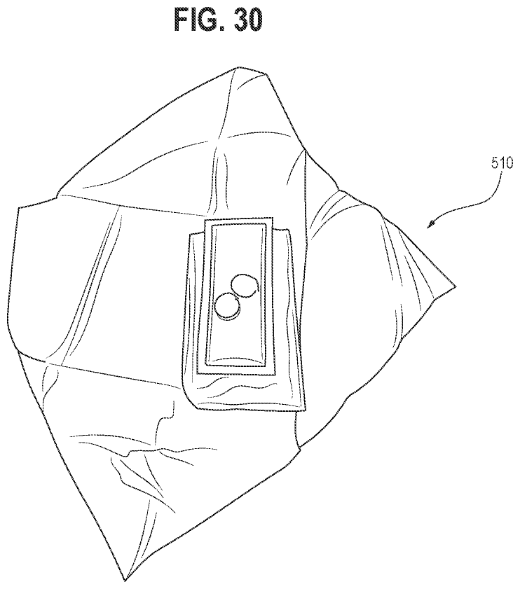

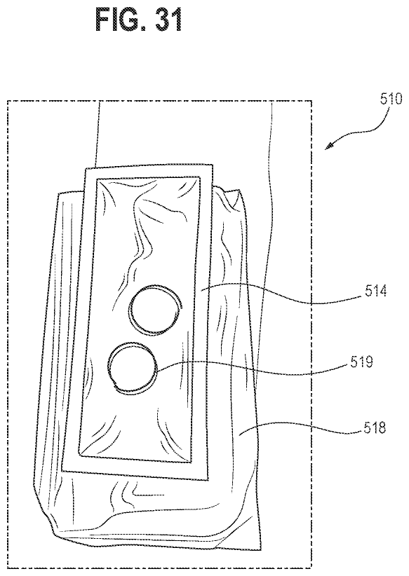

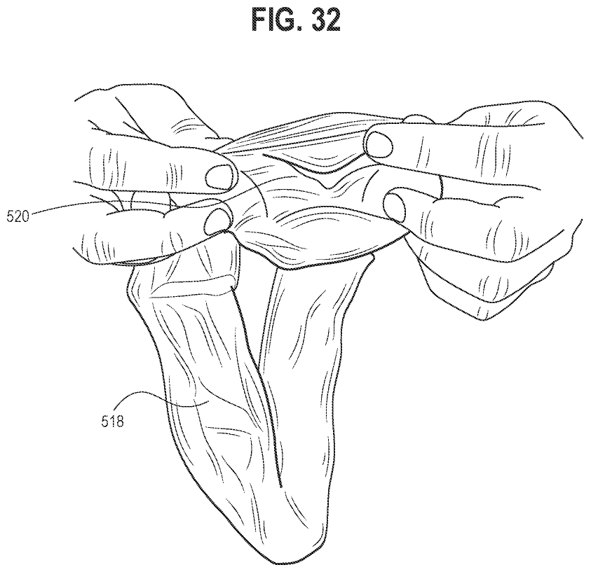

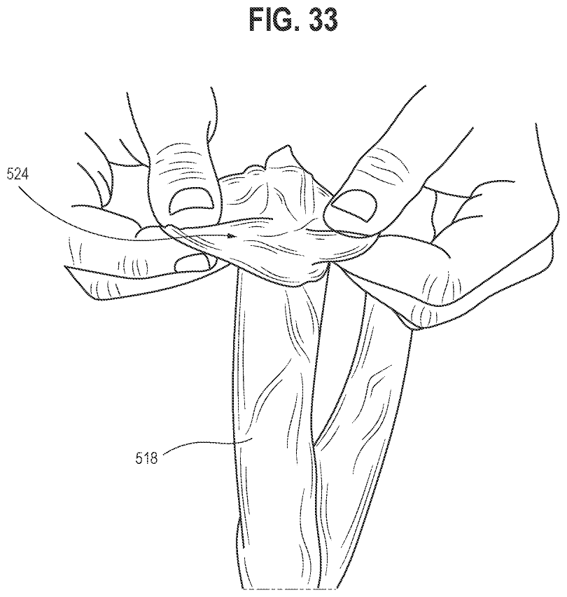

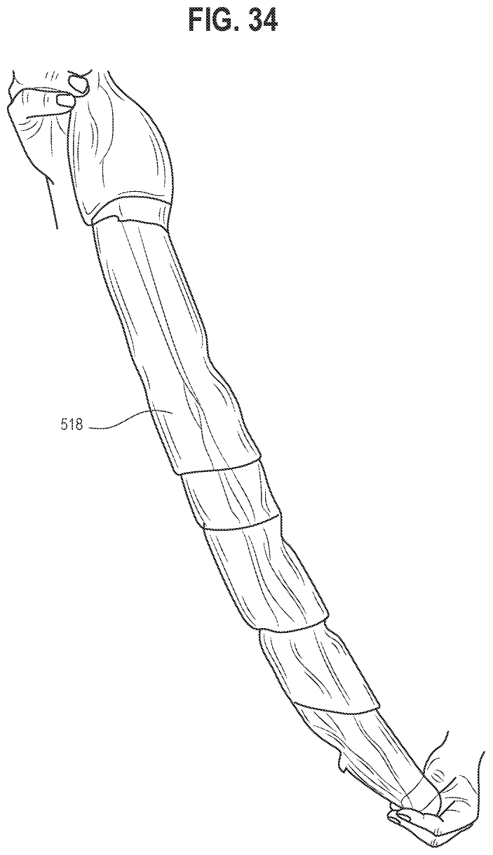

Turning now to FIG. 29, a perspective view of the tray 100 filled with a plurality of medical procedure tools 500 is shown. Included among the medical procedure tools 500 are tools 510 to support an ultrasound procedure. FIG. 30 a perspective view of the unwrapped ultrasound supporting tools 510, and FIG. 31 is a close-up view of the ultrasound supporting tools 510. As seen in FIG. 31, the ultrasound tools 510 can include a transmission gel 514, a sleeve 518, and securing bands 519. As can been seen in FIGS. 32 and 33 respectively, the sleeve 518 can include an opening 520 on a first end and a closed second end 524. Furthermore, the sleeve 518 can extend as shown in FIG. 34 to fit over an ultrasound wand and can be secured on the ultrasound wand using the securing bands 519.

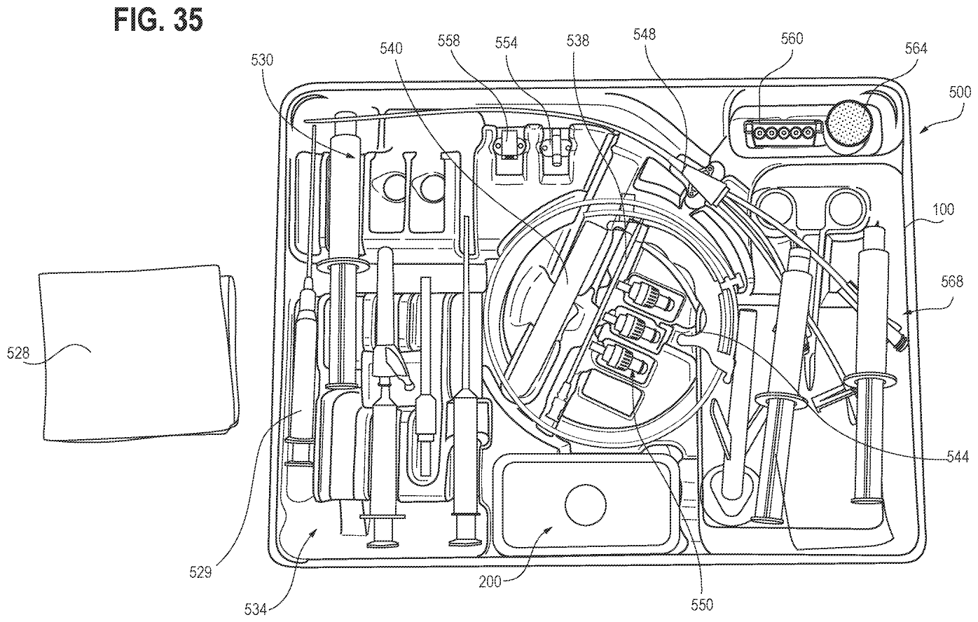

Turning now to FIG. 35 a top view of the tray 100 with the medical procedure tools 500 is shown. The medical procedure tools 500 can include gauze 528, a luer lock syringe 529 for lidocaine with an attached filter straw to draw up lidocaine from a glass ampule. The medical procedure tools 500 also can include incision site preparation tools 530, vein locating tools 534, a vessel dilator 538, a scalpel 540, a catheter insertion tool 544, a central line catheter 548, needle-less connectors 550, a suture loop 554, a box clamp 558, a needle vise 560, a needle receptacle 564, and suture tools 568.

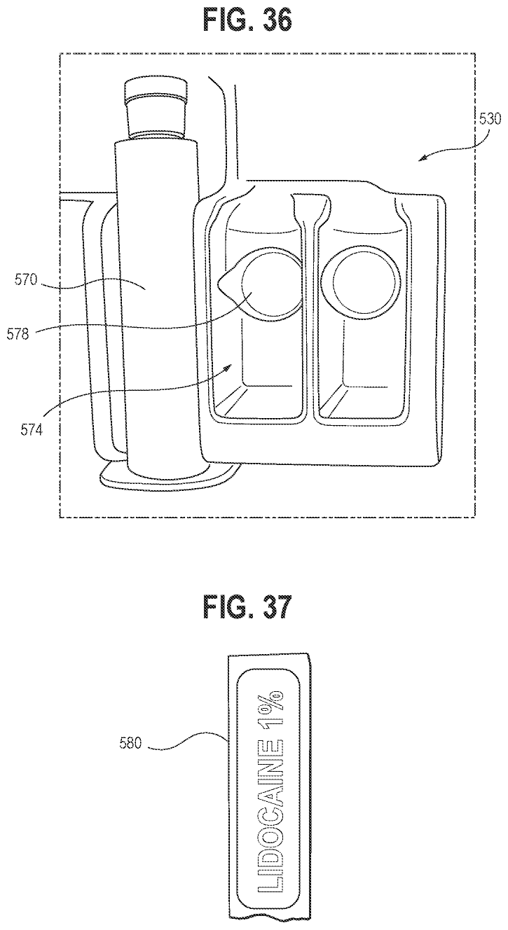

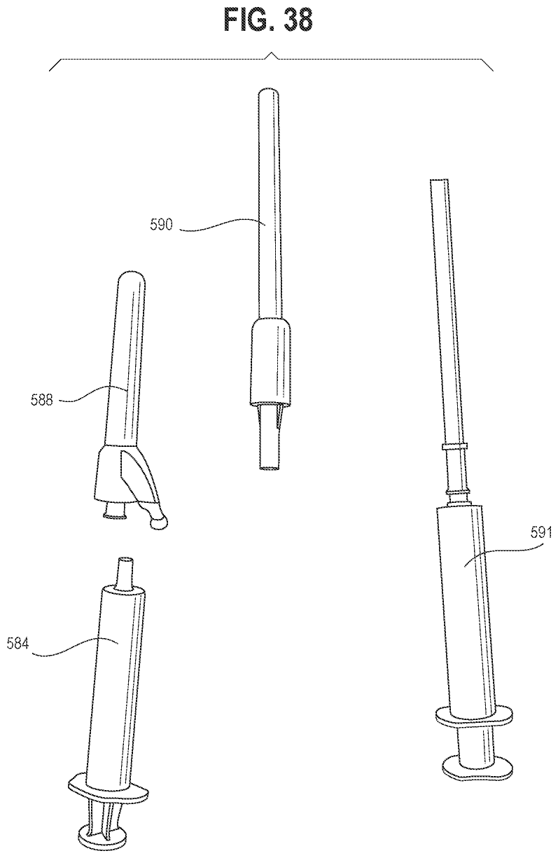

FIG. 36 is a close-up view of the tray 100 and the incision site preparation tools 530. The incision site preparation tools 530 can include a first saline syringe 570 and lidocaine ampules (not shown). In some embodiments, the tray 100 can include compartments 574 sized to hold the lidocaine ampules in a reclining position. The compartments 574 can also include insets 74 for holding the lidocaine ampules in an upright standing position. As shown in FIG. 37, the medical procedure tools 500 can also include a vial content indicator 580.

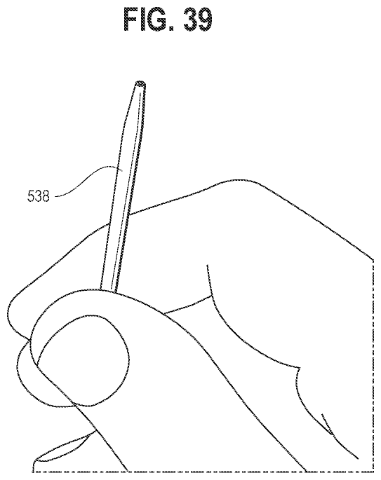

FIG. 38 is a perspective view of the vein locating tools 534. The vein locating tools 534 can include a finder syringe 584, a finder needle 588, a catheter over needle 590, and insertion needle and guiding syringe 591. The vein locating tools 534 can also include a 25 gauge needle for use in injecting lidocaine at the incision site. Together one or more of the vein locating tools 534 can be used to locate and/or begin to open a vein for insertion of a central line catheter. Further, the vessel dilator 538 shown in FIG. 39 can also be used in opening the insertion vein.

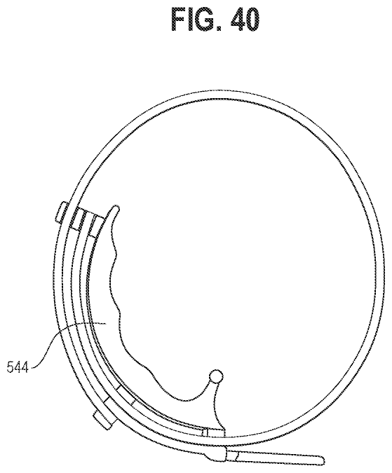

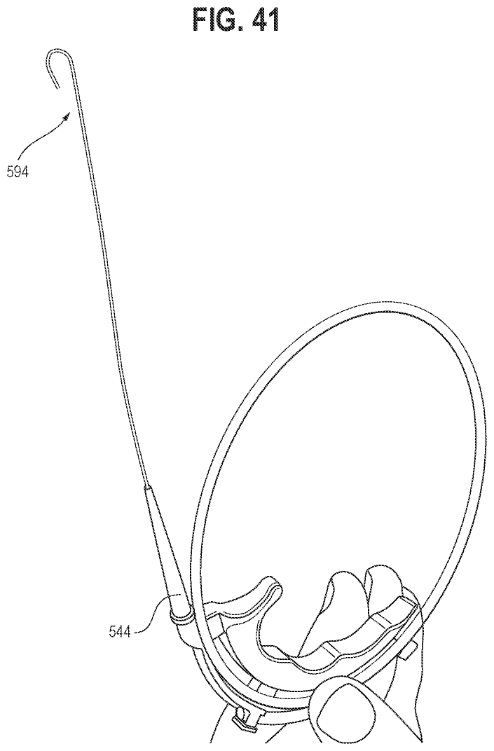

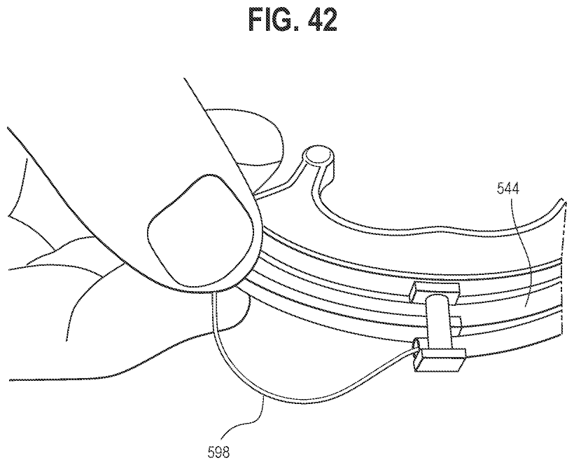

A top view of the catheter insertion tool 544 is shown in FIG. 40. The catheter insertion tool 544 can include a hook 594 and a guide wire 598 as shown in FIGS. 41 and 42 respectively. In operation, the hook 594 and the guide wire 598 can be used to insert the central line catheter 548 into the opened vein.

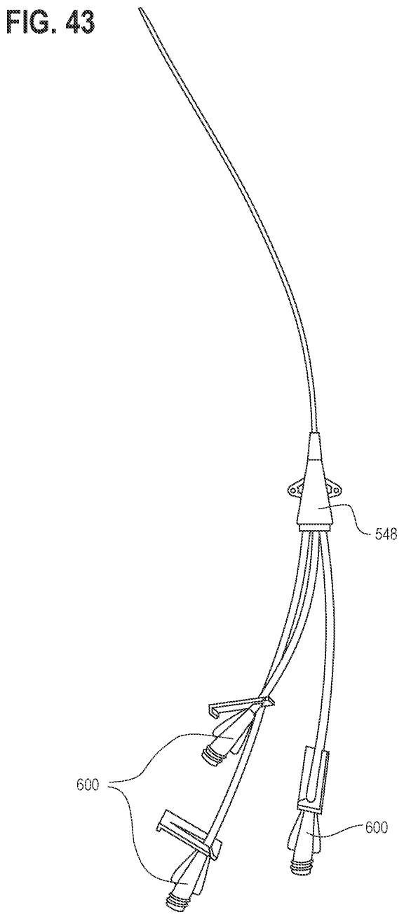

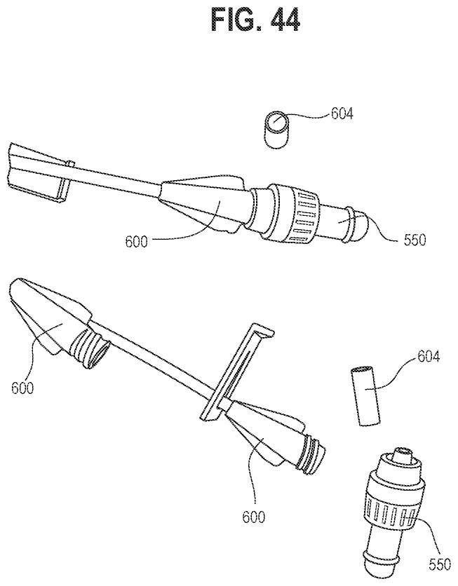

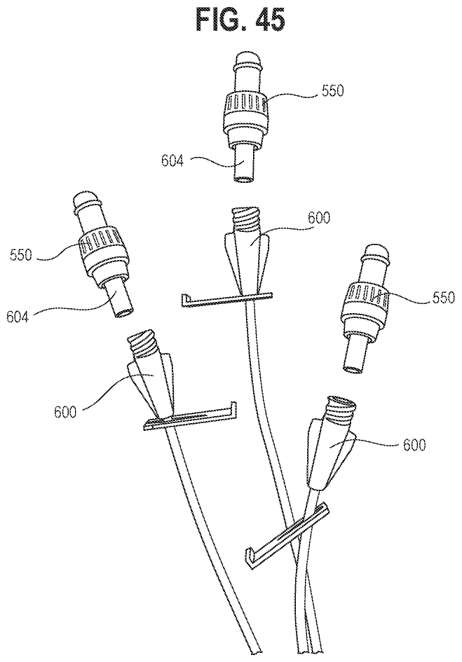

FIG. 43 is a perspective view of the central line catheter 548. As seen in FIG. 43, the central line catheter 548 can include lumens 600 that enable introduction of fluids and medicine into the patient via the central line catheter 548. As seen in FIGS. 44-46, the lumens 600 can couple to the needle-less connectors 550 after a protective sleeve 604 is removed. In some embodiments, the needle-less connectors 550 can be connected to various syringes or medical equipment before being attached to the lumens 600. It should also be noted that additional variation of the central line catheter 548 with more and fewer than 3 lumens 600 are contemplated.

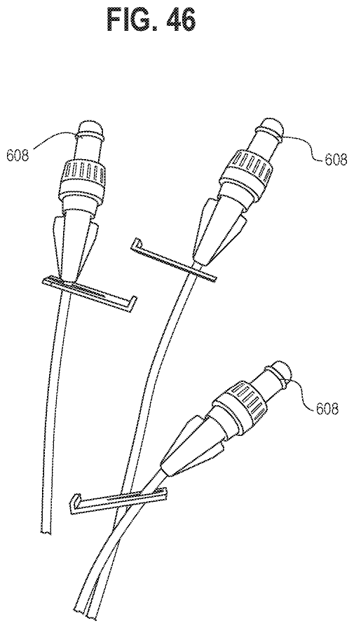

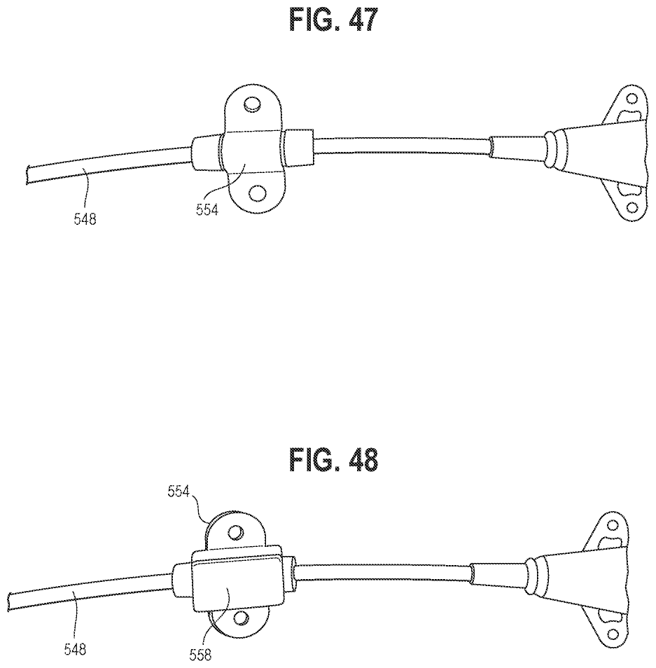

FIG. 47 is a perspective view of the suture loop 554, and FIG. 48 is a perspective view of the box clamp 558. As seen in FIG. 47, when the central line catheter 548 is too long the suture loop 554 can be coupled to the central line catheter 548, and the box clamp 558 can be coupled over the suture loop 554. The suture loop 554 and box claim 558 are sutured to a patient to restrict unintended movement of the central line catheter 548.

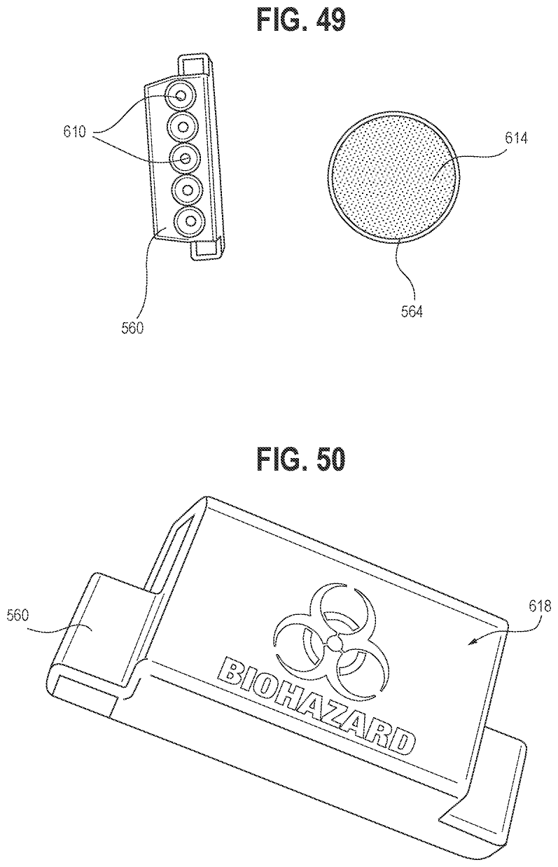

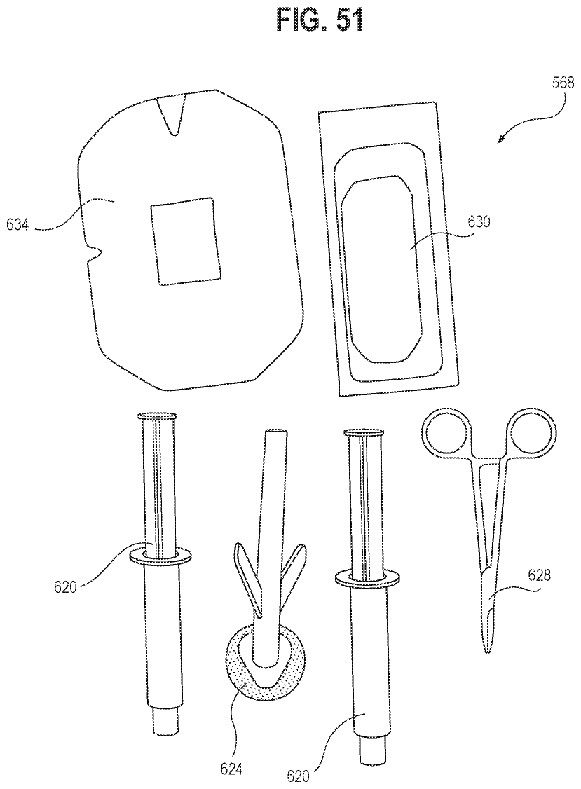

FIG. 49 is top view of the needle vise 560 and the needle receptacle 564. The needle vise 560 can include needle receptacles 610 for containing used needles from the procedure performed with the medical procedure kit 300. Similarly, the needle receptacle 564 can include a foam area 614 to temporarily hold the needles when they are in use during the procedure. As seen in FIG. 50, the needle vise 560 can include a biohazard indicator 618. Additionally, the needle vise 560 and the needle receptacle 564 can be similarly colored to the medical waste basin 200 to indicate the need to dispose of those components with a specialized biohazard disposal process.

As seen in FIG. 51, the suture tools 568 can include additional saline syringes 620, an additional cleansing swab 624, a needle drawer device 628, suture thread 630, and a dressing 634 to be placed over the insertion site.

The embodiments described herein are also directed to a method of using the medical procedure kit 300. The method includes first, unwrapping the tray 100 and the medical procedure tools 500 from the sterile wrap 320. Then, a subset of the medical procedure tools 500 are removed from the plurality of compartments 110. Next, the subset of the medical procedure tools 500 are used to perform a medical procedure, such as preparing to and inserting the central line catheter 548 into a patient. Further, the method can include expelling biological material that accumulated in one or more of the medical procedure tools 500 into the medical waste basin 200. For example, any blood or other fluid that accumulates into any of the various syringes in the incision site preparation tools 530 or vein locating tools 534 can be injected into the medical waste basin 200. Furthermore, in some embodiments, the method can include placing the gauze 528 into the interior region 212 of the basin 200 to soak up biological material and limit potential for the biological materials to spill from the medical waste basin 200. Finally, the method can include removing the medical waste basin 200 from the tray 100 through the access opening 129 and depositing the medical waste basin 200 in a biohazard disposal location remote of the tray 100.

In some embodiment of the method, the subset of the medical procedure tools 500 can be removed in a sequenced order dictated by the medical procedure. The sequenced order can be indicated by a layout of the plurality of compartments 110 that contain the subset of the plurality of medical procedure tools 500. For example, the practitioner can work from the left to the right of the tray 100.

The location of the first compartment 111 for the medical was basin 200 can be selected so that the procedure tools 500 containing medical waste can be deposited in the medical waste basin 200 without unintentionally depositing the waste on other areas of the tray 100. This would require the whole tray 100 to disposed of properly and not just the medical waste basin 200. More specifically, for example, the first compartment 111 can be located at the perimeter of the tray 100 and also can be disposed centrally along one side of the tray 100, as illustrated in FIGS. 1 and 13.

It will be understood that various changes in the details, materials, method steps, and arrangements of parts and components, which have been herein described and illustrated in order to explain the nature and use of the medical procedure kit, tray, and/or the medical waste basin may be made by those skilled in the art within the principle and scope of the subject matter as expressed in the appended claims. Furthermore, while various features have been described with regard to a particular embodiment or a particular approach, it will be appreciated that features described for one embodiment also may be incorporated with the other described embodiments.

What is claimed is:

1. A medical procedure kit, comprising:
a tray defining a plurality of compartments;
a medical waste basin removably supported within a first compartment of the plurality of compartments; and
a plurality of medical procedure tools located within other compartments of the plurality of compartments.

2. The medical procedure kit of claim 1 wherein the medical waste basin defines a gap between at least a position of the first compartment and a bottom of the medical waste basin when the medical waste basin is supported within the first compartment.

3. The medical procedure kit of claim 1, wherein the first compartment defines an access opening to manually engage the medical waste basin.

4. The medical procedure kit of claim 1 further comprising a lid removably attached to the medical waste basin.

5. The medical procedure kit of claim 4 wherein the lid includes a first lip, and the medical waste basin includes a second lip, the first lip mates with the second lip to removeably attach the lid to the medical waste basin.

6. The medical procedure kit of claim 4 wherein the lid defines an aperture sized to accommodate at least a portion of at least one of the plurality of medical procedure tools.

7. The medical procedure kit of claim 5, wherein the lid includes a section that slopes toward the aperture.

8. The medical procedure kit of claim 3 wherein the medical waste basin includes a bottom and a plurality of sidewalls extending from the bottom, and a first side wall of the plurality of sidewalls is shorter than all other of the plurality of sidewalls and is located opposite the access opening.

9. The medical procedure kit of claim 1 wherein the medical waste basin comprises a plurality of protrusions that engage with side walls of the first compartment, to secure the medical waste basin within the tray.

10. The medical procedure kit of claim 9 wherein the plurality of protrusions are positioned under a lip and at corners of the medical waste basin.

11. The medical procedure kit of claim 3 wherein the access opening comprises a passage extending from the first compartment to a neighboring compartment of the plurality of compartments.

12. The medical procedure kit of claim 3 wherein the access opening comprises a lateral recess that enables access to the medical waste basin through a top of the tray.

13. The medical procedure kit of claim 1 wherein the plurality of medical procedure tools includes gauze and the medical waste basin is sized to receive the gauze.

14. The medical procedure kit of claim 1 further comprising a sterile wrap that encloses the tray, the medical waste basin, and the plurality of medical procedure tools.

15. The medical procedure kit of claim 14 further comprising at least one medical procedure guidance indicator located on an exterior of the sterile wrap.

16. The medical procedure kit of claim 1 wherein the plurality of medical procedure tools includes a sterile drape having a treatment area opening and a plurality of medical procedure guidance indicators located around the treatment area opening.

17. The medical procedure kit of claim 1 wherein the plurality of medical procedure tools includes one or more of the following: a luer lock syringe with a filter straw, a prefilled sterile saline syringe, gauze, a 25 gauge needle, a finder needle, a vial or ampule of lidocaine, a catheter over needle, a guiding syringe with an insertion needle, a vessel dilator, a box clamp, a suture loop, a needle vise, a needle receptacle, needle-less connectors, a central line catheter guide wire, a central line catheter, suturing tools, and an ultrasound procedure kit.

18. A method for using a medical procedure kit, the method comprising:
unwrapping a tray of the medical procedure kit from a sterile wrap, the tray defining a plurality of compartments, a medical waste basin supported within a first compartment of the plurality of compartments, and a plurality of medical procedure tools located within other compartments of the plurality of compartments;
removing a subset of the plurality of medical procedure tools from the plurality of compartments;
performing a medical procedure using the subset of the plurality of medical procedure tools; and
expelling biological material that accumulated in one or more of the subset of the plurality of medical procedure tools into the medical waste basin.

19. The method of claim 18 further comprising:
removing the medical waste basin from the tray through an access opening in the tray; and
depositing the medical waste basin in a disposal location remote of the tray.

20. The method for using a medical procedure kit of claim 18 wherein the medical procedure includes opening an incision for a central line catheter and wherein removing the subset of the plurality of medical procedure tools from the plurality of compartments includes removing the subset of the plurality of medical procedure tools in a sequenced order dictated by the medical procedure, wherein the sequenced order is indicated by the plurality of compartments that contain the subset of the plurality of medical procedure tools.

21. The method for using a medical procedure kit of claim 18 wherein expelling the biological material into the medical waste basin includes placing a tip portion of a syringe of the subset of the plurality of medical procedure tools into an aperture defined in a lid of the medical waste basin and depressing a plunger of the syringe.

22. A medical waste basin comprising:
a body sized to be removably nested and supported within a compartment of a tray of a medical procedure kit; and
a lid removably attached to the body.

23. The medical waste basin of claim 22 wherein the lid defines an aperture formed to receive at least a portion of at least one of a plurality of medical procedure tools located within other compartments of the tray.

24. The medical waste basin of claim 22 wherein the body includes a bottom and a plurality of sidewalls extending from the bottom, wherein a first side wall of the plurality of sidewalls is shorter than all other of the plurality of sidewalls and is located opposite an access opening of the tray.
